(19) United States
(12) Patent Application Publication
(10) Pub. No.: US 2020/0360108 A1
(43) Pub. Date: Nov. 19, 2020

(54) CALCIUM SILICATE BASED DENTAL FILLING MATERIAL

(71) Applicant: SONENDO, INC., Laguna Hills, CA (US)

(72) Inventors: Zaza Gomurashvili, Tustin, CA (US); David Tobia, Laguna Hills, CA (US); Stephanie Pascua, Lake Forest, CA (US); Mehrzad Khakpour, Laguna Hills, CA (US); Bjarne Bergheim, Laguna Hills, CA (US)

(21) Appl. No.: 16/875,193

(22) Filed: May 15, 2020

Related U.S. Application Data
(60) Provisional application No. 62/849,749, filed on May 17, 2019.
(60) Provisional application No. 62/861,242, filed on Jun. 13, 2019.

(51) Int. Cl. A61C 5/50; A61K 6/853

(52) U.S. Cl. CPC A61C 5/50; A61K 6/853

(57) ABSTRACT

A curable mixture and method of using the mixture are disclosed. In some embodiments, the mixture comprises a calcium silicate compound, a filler material, a non-aqueous carrier liquid, and a secondary carrier liquid, and has properties suitable for use as a tooth filling after curing.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims priority to U.S. Provisional Patent Application No. 62/849,749 filed May 17, 2019, and to U.S. Provisional Patent Application 62/861,242 filed Jun. 13, 2019, the contents of each of which are incorporated by reference herein in their entirety for all purposes.

### BACKGROUND

In conventional endodontic procedures, an opening is drilled through the crown of a diseased tooth, and endodontic files are inserted into the root canal system to open the canal spaces and remove organic material therein. The root canal is then filled with solid matter such as gutta percha and an obturation material, and the tooth is restored. However, this procedure will not remove all organic material from the canal spaces, which can lead to post-procedure complications such as infection. In addition, motion of the endodontic file may force organic material through an apical opening into periapical tissues. In some cases, the end of the endodontic file itself may pass through the apical opening. Such events may result in trauma to the soft tissue near the apical opening and lead to post-procedure complications.

Current treatment techniques for tooth decay (caries) generally include mechanical removal of the caries and diseased tissue (e.g., using dental burs, excavators, etc.), which will expose healthy dentin. However, the bur (or other mechanical instrument) may not differentiate between diseased and healthy dentin, and other instruments such as excavators and explorers may not be able to accurately determine the extent to which tooth removal should continue. This may result in either incomplete removal of caries or overly aggressive removal of healthy dentin, which may in turn reduce the longevity of the tooth. The removed portions of the tooth can then be filled with solid matter such as composite, resin, gold, porcelain, etc., and the tooth can be restored. However, this procedure may not remove all decayed material from the tooth, which combined with inadequate penetration of the restorative material can result in bacterial leakage and subsequently post-procedure complications such as infection or recurrent caries. In part to minimize the risk of reinfection, endodontic material placement typically requires the use of a gutta percha point to encourage penetration of the obturation material into lateral canals and isthmi. In addition, the use of a dental drill and anesthetics may be uncomfortable for the patient. Various filling spaces within or adjacent to a tooth can benefit from improvements in dental treatment techniques. Examples of such filling spaces include but are not limited to root canals, cavities resulting from the removal of caries, other openings such as cracks and gaps, and/or missing portions of teeth (e.g., resulting from fracture and/or wear). Accordingly, it can be advantageous to provide improved compositions, methods and apparatus for treating dental decay.

More recently, dental apparatuses have been developed that can deliver a curable mixture to a treatment region without the necessity of an obturation point. (See U.S. Pat. No. 9,877,801, the entire contents of which are incorporated herein by reference for all purposes). Various formulations are known that can be used as curable mixtures. However, the compatibility of current materials with the new technology is less than desired. Thus, the need for more advanced obturation materials is needed.

### SUMMARY

In one aspect, a curable mixture of ingredients is disclosed. The curable mixture includes (a) a calcium silicate compound; (b) a filler material; (c) a non-aqueous carrier liquid; and (d) a secondary carrier liquid different than the non-aqueous carrier liquid.

In some embodiments, the calcium silicate compound is selected from at least one of calcium silicate, dicalcium silicate and tricalcium silicate. In some embodiments, the non-aqueous carrier liquid comprises at least one of acetic acid, acetone, acetonitrile, 1-butanol, 2-butanone, ethyl acetate, methanol, ethanol, propanol, butanol, dimethyl sulfoxide, dimethylformamide, 1,4-dioxane, methyl isocyanide, pyridine, tetrahydrofuran, ethylene glycol, propylene glycol, triethylene glycol, poly(ethylene glycol), poly(propylene glycol) and glycerol. In some embodiments, the non-aqueous carrier liquid comprises at least one of acetic acid, 1-butanol, methanol, ethanol, propanol, butanol, dimethyl sulfoxide, dimethylformamide, ethylene glycol, propylene glycol, triethylene glycol, poly(ethylene glycol), poly(propylene glycol), glycerol and diethylene glycol monomethyl ether. In some embodiments, the secondary carrier liquid comprises at least one of water, acetic acid, acetone, acetonitrile, 1-butanol, 2-butanone, ethyl acetate, methanol, ethanol, propanol, butanol, dimethyl sulfoxide, dimethylformamide, 1,4-dioxane, methyl isocyanide, pyridine, tetrahydrofuran. In some embodiments, the secondary carrier liquid comprises at least one of water, acetic acid, 1-butanol, methanol, ethanol, propanol, butanol and dimethyl sulfoxide. In some embodiments, the curable mixture further comprises an X-ray radiopaque material. In some embodiments, the curable mixture further comprises a phosphate salt. In some embodiments, the curable mixture is provided in two parts.

In some embodiments, a method of preparing an obturation material comprising forming a reaction mixture comprising the curable mixture under conditions suitable to form the obturation material. In some embodiments, the reaction mixture forms the obturation material upon exposure to water and/or moisture. In some embodiments, the calcium silicate compound is calcium trisilicate. In some embodiments, the curable mixture of ingredients comprises less than 20 wt % calcium silicate, based on the total weight of the curable mixture. In some embodiments, the curable mixture of ingredients comprises between 0.1 wt. % to 30 wt. % filler, based on the total weight of the curable mixture of ingredients. In some embodiments, the curable mixture of ingredients comprises between 0.1 wt. % to 3 wt. % metal oxide as a filler, based on the total weight of the curable mixture of ingredients. In some embodiments, the filler comprises fumed silica. In some embodiments, the curable mixture of ingredients comprises between 40 wt % and 60 wt. % of a secondary carrier liquid. In some embodiments, the secondary carrier liquid is water. In some embodiments, the secondary carrier liquid further comprises a radiopaque material. In some embodiments, the secondary carrier liquid comprises a water-soluble radiopaque material.

In some embodiments, a method of filling a tooth is disclosed. The method includes identifying a tooth having a cavity in need of filling, positioning the curable mixture within the cavity, and curing the curable mixture within the cavity.

In some embodiments, a method of filling a root canal is disclosed. The method includes identifying a tooth having a root canal in need of filling, positioning the curable mixture within the root canal, and curing the curable mixture within the root canal.

In some embodiments, the curable mixture is positioned using a pressure wave generator.

In some embodiments, a method of filling a root canal is disclosed that comprises:

providing a first flow of a first part of a two-part curable composition, wherein the first part comprises:

(a) a calcium silicate compound,

(b) a filler material, and

(c) a first carrier liquid;

providing a second flow of a second part of the two-part curable composition, wherein the second part comprises a second carrier liquid; combining the first and second flow to form a final curable composition; and positioning the final curable composition within the root canal.

In some embodiments, the first and second carrier liquids are the same. In other embodiments, the first and second carrier liquids are different. In some embodiments, the first carrier liquid is a non-aqueous liquid. In some embodiments, the second carrier liquid is an aqueous liquid.

Some embodiments disclosed include a kit that comprises a first container comprising a first part of a two-part curable composition, wherein the first part comprises:

(a) a calcium silicate compound,

(b) a filler material, and

(c) a first carrier liquid; and

a second container comprising a second part of the two-part curable composition, wherein the second part comprises a second carrier liquid.

In some embodiments, the first and second carrier liquids are the same. In other embodiments, the first and second carrier liquids are different. In some embodiments, the first carrier liquid is a non-aqueous liquid. In some embodiments, the second carrier liquid is an aqueous liquid.

In some embodiments, a method of filling a root canal is disclosed that comprises providing a first flowable part of a two-part curable composition, wherein the first part comprises:

(a) a calcium silicate compound,

(b) a filler material, and

(c) a first carrier liquid;

providing a second flowable part of the two-part curable composition, wherein the second part comprises a second carrier liquid; combining the first and second flowable parts to form a final curable composition; and positioning the final curable composition within the root canal. In some embodiments, the first and second carrier liquids are the same. In other embodiments, the first and second carrier liquids are different. In some embodiments, the first carrier liquid is a non-aqueous liquid, and the second carrier liquid is an aqueous liquid.

Some embodiments include a kit that comprises a two-part curable composition, wherein a first container comprises a first part that comprises:

(a) a calcium silicate compound,

(b) a filler material, and

(c) a first carrier liquid; and

a second container comprising a second part of the two-part curable composition, wherein the second part comprises a second carrier liquid.

In some embodiments, the first and second carrier liquids are the same. In other embodiments, the first and second carrier liquids are different. In some embodiments, the first carrier liquid is a non-aqueous liquid and the second carrier liquid is an aqueous liquid. In some embodiments, the first part is a paste and the second part is a liquid. In other embodiments, the first part of the curable mixture is introduced into an application device as a paste, and the second part is introduced into the application device as a liquid, prior to mixing the first and second parts to form the curable mixture and dispensing the curable mixture into a space in a tooth. In some embodiments, the second part comprises a radiopaque material. In some embodiments, the first part comprises a flow rate of about 50 g/min. to about 500 g/min. at 20 psi. In some embodiments, the second part comprises a viscosity between about 0.1 cps and 20 cps at 25° C.

In another aspect, a curable mixture of ingredients is described. The curable mixture of ingredients are provided in two parts comprising a first part comprising a flowable mixture comprising:

(a) a calcium silicate;

(b) a filler material; and

(c) a non-aqueous carrier liquid; and

a second part comprising an aqueous carrier liquid.

In some embodiments, the first part is a paste. In some embodiments, a total weight percent of the non-aqueous carrier liquid and the aqueous carrier liquid is about 45 wt % to about 60 wt %. In some embodiments, the non-aqueous carrier liquid comprises propylene glycol. In some embodiments, the non-aqueous carrier liquid comprises poly(ethylene glycol). In some embodiments, the curable mixture comprises 1 wt % to 20 wt % tricalcium silicate. In some embodiments, the curable mixture comprises 1 wt % to 15 wt % tricalcium silicate. In some embodiments, the curable mixture comprises 7 wt % to 13 wt % tricalcium silicate. In some embodiments, the calcium silicate consists essentially of tricalcium silicate. In some embodiments, the curable mixture comprises less than 40 wt. % filler. In some embodiments, the curable mixture comprises between 0.1 wt. % to 30 wt. % filler. In some embodiments, the filler comprises a metal oxide. In some embodiments, the curable mixture comprises 0.1 wt. % to 10 wt. % fumed silica. In some embodiments, the curable mixture comprises 0.2 wt. % to 2 wt. % fumed silica. In some embodiments, the first part comprises a radiopaque compound. In some embodiments, the first part comprises 10 wt. % to 40 wt. % of a radiopaque compound. In some embodiments, the first part comprises 15 wt. % to 40 wt. % of a radiopaque compound. In some embodiments, the radiopaque compound in the first part comprises ytterbium fluoride. In some embodiments, the curable mixture comprises 10 wt. % to 30 wt. % of ytterbium fluoride. In some embodiments, the second part comprises a radiopaque compound. In some embodiments, the aqueous carrier liquid of the second part comprises a water-soluble radiopaque compound. In some embodiments, the second part comprises 5 wt. % to 20 wt. % of a radiopaque material. In some embodiments, the second part comprises potassium iodide as a radiopaque compound. In some embodiments, the second aqueous carrier liquid has a viscosity of about 1 cps to about 30 cps at 25° C. In some embodiments, the second aqueous carrier liquid has a viscosity between about 0.1 cps and 20 cps at 25° C. In some embodiments, the first part and second part of the two-part curable mixture are combinable to initiate a curing process. In some embodiments, the first part has a flow rate of about 50 g/min. to about 500 g/min. at 20 psi.

In some embodiments, a method of filling a root canal of a tooth with the curable mixture is described. The method comprises: obtaining a liquid jet device for delivering the curable mixture to the root canal of the tooth comprising a first supply line, a second supply line, a mixing chamber and a nozzle; positioning the liquid jet device near a treatment region of the tooth; supplying the first part of the two-part curable mixture to the second supply line of a liquid jet device; supplying the second part of the two-part curable mixture to the liquid jet device through the first supply line and forming a liquid jet from the second part of the two-part curable mixture; mixing the first and second parts of the two-part curable mixture in the mixing chamber to form a reaction mixture; filling the root canal with the reaction mixture; and curing the reaction mixture to form an obturation material within the root canal.

In some embodiments, a method of preparing an obturation material is described. The method comprises forming a reaction mixture comprising the curable mixture under conditions suitable to form the obturation material.

In some embodiments, a method of filling a root canal is described. The method comprises: identifying a tooth having a root canal in need of filling; positioning the curable mixture within the root canal; and curing the curable mixture within the root canal. In some embodiments, the curable mixture is positioned using a pressure wave generator.

In some embodiments, a method of filling a tooth is described. The method comprises: identifying a tooth having a cavity in need of filling; positioning the curable mixture within the cavity; and curing the curable mixture within the cavity.

In another aspect, a method of filling a root canal is described. The method comprises obtaining a first flowable part of a two-part curable composition that comprises:

(a) a calcium silicate compound,

(b) a filler material, and

(c) a first carrier liquid;

obtaining a second flowable part of the two-part curable composition, wherein the second part comprises a second carrier liquid; combining the first and second flowable parts to form a final curable composition; and positioning the final curable composition within the root canal.

In some embodiments, the first and second carrier liquids are the same. In some embodiments, the first and second carrier liquids are different. In some embodiments, the first carrier liquid is a non-aqueous liquid. In some embodiments, the second carrier liquid is an aqueous liquid.

These and other embodiments are described in greater detail below.

### BRIEF DESCRIPTION OF THE DRAWINGS

The foregoing and other features, aspects, and advantages of the embodiments of the apparatus, compositions and methods of filling spaces in teeth are described in detail below with reference to the drawings of various embodiments, which are intended to illustrate and not to limit the embodiments of the invention. The drawings comprise the following figures in which:

FIG. 1A is a schematic diagram of a dental treatment system for treating a root canal, according to various embodiments disclosed herein.

FIG. 1B is a schematic diagram of a system that includes components configured to clean unhealthy or undesirable material from a treatment region on an exterior surface of the tooth.

FIG. 1C is a schematic diagram of the system of FIG. 1B, in which the system is configured to fill a treated carious region of the tooth.

FIG. 2A is a schematic top plan view of a delivery device that can be used to combine a first composition with a second composition to form a curable mixture and to fill a treatment region.

FIG. 2B is a schematic side sectional view of a portion of the delivery device of FIG. 2A.

### DETAILED DESCRIPTION

To protect the long-term health of the tooth, it can be advantageous to substantially fill the filling space or spaces of a tooth created from removal of caries, root canal treatment, and/or natural wear. When the restoration follows a root canal treatment it can be important to fill not only the major canal spaces, but also any minor cracks and open spaces in the tooth with the filling material. Similarly, when the restoration follows a caries treatment it can be important to fill the resulting dental spaces in order to provide dimensional stability and/or structural integrity to the tooth.

In various embodiments, the filling material is an obturation material. The term “obturation material” refers to a material that is configured to fill root canals, restore carious lesions, and/or modify the surface of the tooth. The obturation material can be a curable or polymerizable restorative composition that includes a curable mixture that is cured or hardened to form the final material, which may be referred to as a cured mixture or “tooth filling.” Indeed, it should be appreciated that terms such as setting, curing, hardening, polymerizing, etc. all refer to processes by which the obturation material components are transformed into the final cured mixture in the tooth. In this context, an obturation material is “suitable for use as a tooth filling” when the corresponding cured tooth filling has properties that meet standards set by an appropriate regulatory body (e.g. ISO 6876). A cured obturation material having such properties is considered to meet the standards regardless of whether the regulatory body has provided official notification to that effect.

In some embodiments, various obturation material compositions or components thereof as described herein can be formed into a coherent collimated jet. For example, in an embodiment, an obturation material composition or components thereof as described herein can be formed into a liquid jet that forms a substantially parallel beam (e.g., is “collimated”) over distances ranging from about 0.01 cm to about 10 cm. In some embodiments, the velocity profile transverse to the propagation axis of the jet is substantially constant (e.g., is “coherent”). For example, in some implementations, away from narrow boundary layers near the outer surface of the jet (if any), the jet velocity is substantially constant across the width of the jet. Therefore, in certain advantageous embodiments, the liquid jet (e.g., as delivered by an apparatus as described herein) may comprise a coherent, collimated jet (a “CC jet”). In some implementations, the CC jet may have velocities in a range from about 100 m/s to about 300 m/s, for example, about 190 m/s in some embodiments. In some implementations, the CC jet can have a diameter in a range from about 5 microns to about 1000 microns, in a range from about 10 microns to about 100 microns, in a range from about 100 microns to about 500 microns, or in a range from about 500 microns to about 1000 microns. Further details with respect to CC jets that can be comprised of obturation material compositions or components thereof as described herein can be found in U.S. Patent Publication No. 2007/0248932, which is hereby incorporated by reference herein in its entirety for all that it discloses or teaches.

In an embodiment, the curable mixture of ingredients comprises:

(a) a calcium silicate compound;

(b) a filler material;

(c) a non-aqueous carrier liquid; and

(d) a secondary carrier liquid different than the non-aqueous carrier liquid.

In another embodiment, the obturation material comprises a two-part curable mixture that comprises a first part and a second part, where the first part comprises:

(a) a calcium silicate compound,

(b) a filler material, and

(c) a first carrier liquid;

and the second part comprises a second carrier liquid. In some embodiments, the first carrier liquid and the second carrier liquid are the same. In other embodiments, the first carrier liquid and the second carrier liquid are different. In some embodiments, the first carrier liquid is a non-aqueous carrier liquid. In some embodiments, the second carrier liquid is an aqueous carrier liquid. In some embodiments, the first part and second part of the two-part curable mixture are combined to initiate the curing process. In some embodiments, the first part and second part of the two-part curable mixture are combined prior to the mixture being introduced, or as the mixture is introduced, into the space or spaces of a tooth created from removal of caries, root canal treatment, and/or natural wear. In some embodiments, the two-part curable mixture is provided as a kit that comprises a first container comprising the first part of the mixture and a second container comprising the second part of the mixture.

Various calcium silicate compounds are suitable for use in the curable mixture of ingredients. In some embodiments, the calcium silicate compound comprises at least one of calcium silicate, dicalcium silicate, or tricalcium silicate. For example, in some embodiments, the calcium silicate compound comprises tricalcium silicate; in some embodiments, the calcium silicate compound consists essentially of tricalcium silicate.

The curable mixture can contain various amounts of the calcium silicate compound. For example, in some embodiments, the amount of the calcium silicate compound (e.g. tricalcium silicate) in the curable mixture is in any one of the following ranges: 1 wt. % to 20 wt. %, 1 wt. % to 18 wt. %, 1 wt. % to 15 wt. %, 1 wt. % to 13 wt. %, 1 wt. % to 12 wt. %, 1 wt. % to 10 wt. %, 5 wt. % to 15 wt. %, 5 wt. % to 13 wt. %, 5 wt. % to 12 wt. %, 5 wt. % to 10 wt. %, 7 wt. % to 15 wt. %, 7 wt. % to 13 wt. %, 7 wt. % to 12 wt. %, 7 wt. % to 10 wt. %, 8 wt. % to 15 wt. %, 8 wt. % to 13 wt. %, 8 wt. % to 12 wt. %, 8 wt. % to 10 wt. %, 10 wt. % to 20 wt. %, 10 wt. % to 18 wt. %, 10 wt. % to 15 wt. %, or 10 wt. % to 13 wt. %, based on total weight of curable mixture. In some embodiments, the curable mixture comprises the calcium silicate compound (e.g. tricalcium silicate) in any one of the amounts within the aforementioned ranges, such as, or about, 1 wt. %, 3 wt. %, 5 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, or 20 wt. %, or any range of values therebetween.

In some embodiments, the calcium silicate compound is in a microparticulate form. In some embodiments, the microparticles have an average particle size of about 5 microns or less, about 3 microns or less, or about 2 microns or less. In some embodiments, the calcium silicate compound is substantially anhydrous.

Fillers can be used to adjust the viscosity and/or rheological properties of the curable mixture. Various filler materials are suitable for use in the curable mixture of ingredients. In some embodiments, the filler material is non-reactive with tooth material and/or the other components of the mixture. For example, in some embodiments, the mixture comprises a non-reactive filler material. In some embodiments, the filler material comprises at least one of an inorganic metal oxide, a metal fluoride, a silicate glass and quartz. In other embodiments, the mixture comprises a filler material comprising at least one of an inorganic material such as ZnO, a bioactive glass, fumed silica and a non-reactive glass. In some embodiments, the filler material is fumed silica. Examples of fumed silica include, but are not limited to, Aerosil OX-50, Aerosil OX-130, Aerosil OX-200, Cab-O-Sil TS530, Cab-O-Sil TS720 and Cab-O-Sil M5, and mixtures thereof. In some embodiments, the filler material is a bioactive glass. In some embodiments, the bioactive glass is a calcium containing glass such as Bioglass. In some embodiments, the filler material is a non-reactive glass. Examples of non-reactive glass, include but are not limited to, bariumaluminosilicate, bariumborosilicate, bariumaluminoborosilicate, strontiumaluminosilicate, strontiumborosilicate, and strontiumaluminoborosilicate, and mixtures thereof. In some embodiments, the non-reactive glass is bariumborosilicate glass. In some embodiments, the filler is a bioceramic material.

Various amounts of filler material can be included in the curable mixtures described herein, depending on the viscosity and/or rheological properties desired. In some embodiments, the curable mixture comprises an amount of a filler material in any one of the following ranges, such as 0.1 wt. % to 35 wt. %, 0.1 wt. % to 30 wt. %, 0.1 wt. % to 25 wt. %, 0.1 wt. % to 20 wt. %, 0.1 wt. % to 10 wt. %, 0.1 wt. % to 5 wt. %, 0.1 wt. % to 3 wt. %, 0.1 wt. % to 2 wt. %, 0.2 wt. % to 3 wt. %, 0.2 wt. % to 2 wt. %, 0.2 wt. % to 1.5 wt. %, 0.5 wt. % to 5 wt. %, 0.5 wt. % to 1.5 wt. %, 1 wt. % to 10 wt. %, 1 wt. % to 8 wt. %, 1 wt. % to 3 wt. %, or 2 wt. % to 6 wt. %, filler material, based on the total weight of the curable mixture. For example, in some embodiments, the curable mixture comprises a filler material in an amount of, or of about, 0.1 wt. %, 0.4 wt. %, 0.5 wt. %, 0.6 wt. %, 0.8 wt. %, 1 wt. %, 2 wt. %, 3 wt. %, 4 wt. %, 5 wt. %, 6 wt. %, 7 wt. %, 10 wt. %, 15 wt. %, 20 wt. %, 25 wt. %, 27 wt. % or 30 wt. % filler material, based on the total weight of the curable mixture, or any range of values therebetween.

In some embodiments, the filler material is in a microparticulate form. In some embodiments, the microparticles have an average particle size of about 5 microns or less, about 3 microns or less, or about 2 microns or less. In some embodiments, the filler material is substantially anhydrous.

Various hygroscopic materials are suitable for use in the curable mixture of ingredients. Examples of hygroscopic materials include, but are not limited to magnesium sulfate, calcium chloride, copper sulfate, or a mixture thereof. In some embodiments, the hygroscopic material is magnesium sulfate. Various amounts of hygroscopic material can be included in the curable mixtures described herein.

n some embodiments, the curable mixture comprises an amount of a hygroscopic material in any one of the following ranges, such as 0.1 wt. % to 15 wt. %, 0.5 wt. % to 10 wt. %, 0.1 wt. % to 5 wt. %, 0.5 wt. % to 12 wt. %, 1 wt. % to 12 wt. %, 0.1 wt. % to 3 wt. %, 1 wt. % to 10 wt. %, 2 wt. % to 6 wt. %, or 1 wt. % to −8 wt. % hygroscopic material, based on the total weight of the curable mixture. For example, in some embodiments, the curable mixture comprises a hygroscopic material in an amount of, or of about, 0.1 wt. %, 0.4 wt. %, 0.5 wt. %, 0.6 wt. %, 0.8 wt. %, 1 wt. %, 2 wt. %, 3 wt. %, 4 wt. %, 5 wt. %, 6 wt. %, 7 wt. %, 10 wt. % or 12 wt. % hygroscopic material, or any range of values therebetween. In some embodiments, the hygroscopic material is in a microparticulate form. In some embodiments, the microparticles have an average particle size of about 5 microns or less, about 3 microns or less, or about 2 microns or less. In some embodiments, the hygroscopic material is substantially anhydrous.

Various X-ray radiopaque material are suitable for use in the curable mixture of ingredients. In some embodiments, the X-ray radiopaque material comprises one or more X-ray radiopaque elements or materials. Examples of X-ray radiopaque elements include, but are not limited to, Yb, Ba, Bi, W, Sr, Zr or a mixture thereof. In some embodiments, the X-ray radiopaque materials include, but is not limited to, calcium iodide, potassium iodide, YbF₃, ZrO₂, BaF₂, BaSO₄, SrSO₄, Sr₃(PO₄)₂, BaWO₄, CaWO₄ and SrWO₄. In some embodiments, the X-ray radiopaque material is YbF₃. In other embodiments, the X-ray radiopaque material is calcium iodide or potassium iodide. The radiopaque material may be water-soluble, such as a water-soluble radiopaque monomer or a water-soluble radiopaque salt. In some embodiments, the water-soluble radiopaque material may be iodophenyl functionalized polyethylene glycol monomer, water-soluble iodide or barium salt, such as calcium iodide, potassium iodide, sodium iodide, or barium chloride. In other embodiments, radiopaque salts may include (MRI) radio-contrast agents such as a gadolinium salt and/or a sodium diatrizoate type agent (such as sodium diatrizoate hydrate). Other radiopaque materials include, but are not limited to, radiopaque aromatic acids, such as a water soluble radiopaque aromatic acid derived (meth)acrylate, 5-acrylamido-2,4,6-triiodo isophthalic acid, or diatrizoate sodium hydrate.

Various amounts of the X-ray radiopaque material can be included in the curable mixture. The amount may be selected to render the resulting cured mixture X-ray radiopaque as defined by the International Standards Organization (e.g., ISO 6876:2012), and in some embodiments, the curable mixture has a radiopacity greater than 1 mmAl, or greater than 2 mmAl, or greater than 3 mmAl. In some embodiments, the curable mixture comprises an amount of an X-ray radiopaque material in any one of the following ranges, such as 10 wt. % to 40 wt. %, 10 wt. % to 38 wt. %, 10 wt. % to 35 wt. %, 10 wt. % to 30 wt. %, 10 wt. % to 22 wt. %, 15 wt. % to 40 wt. %, 15 wt. % to 35 wt. %, 15 wt. % to 30 wt. %, 15 wt. % to 25 wt. %, 18 wt. % to 40 wt. %, 18 wt. % to 38 wt. %, 18 wt. % to 35 wt. %, 18 wt. % to 30 wt. %, 18 wt. % to 22 wt. %, 25 wt. % to 40 wt. %, 25 wt. % to 38 wt. %, or 25 wt. % to 35 wt. %, based on the total weight of the curable mixture. For example, in some embodiments, the curable mixture comprises an amount of an X-ray radiopaque material within one or more of the aforementioned ranges, such as, or about, 10 wt. %, 18 wt. %, 19 wt. %, 20 wt. %, 21 wt. %, 22 wt. %, 24 wt. %, 25 wt. %, 26 wt. %, 28 wt. %, 30 wt. %, 32 wt. %, 35 wt. %, 38 wt. % or 40 wt. % X-ray radiopaque material, or any range of values therebetween.

n some embodiments, both the first part and the second part of the two-part curable mixture comprise a radiopaque material. The curable mixture may comprise first and second radiopaque materials that are the same or different. In some embodiments, a first part of the curable mixture comprising a non-aqueous carrier liquid comprises a first radiopaque material, and a second part of the curable mixture comprising an aqueous carrier liquid comprises a second radiopaque material that is water-soluble.

In some embodiments, the X-ray radiopaque material is in a microparticulate form. In some embodiments, the microparticles have an average particle size of about 5 microns or less, about 3 microns or less, or about 2 microns or less. In some embodiments, the X-ray radiopaque material is substantially anhydrous.

A carrier liquid or fluid can dissolve and/or suspend the other ingredients of the curable mixture, so that the curable mixture can be more conveniently applied to a tooth. In some embodiments, the carrier liquid is water soluble. In some embodiments, the carrier liquid is water miscible. In some embodiments, the carrier liquid is substantially anhydrous. The curable mixture can contain a variety of carrier liquids or mixtures of carrier liquids (e.g. non-aqueous carrier liquids and secondary carrier liquids).

Various non-aqueous carrier liquids are suitable for use in the curable mixture of ingredients (for example, as the first carrier liquid in a two-part curable composition). In some embodiments, the non-aqueous carrier liquid comprises a water soluble or water miscible carrier liquid. In some embodiments, the non-aqueous carrier liquid comprises at least one of acetic acid, acetone, acetonitrile, 1-butanol, 2-butanone, ethyl acetate, methanol, ethanol, propanol, butanol, dimethyl sulfoxide, dimethylformamide, 1,4-dioxane, methyl isocyanide, pyridine, tetrahydrofuran, and a polyol. Examples of polyols include, in some embodiments, ethylene glycol, propylene glycol, triethylene glycol, diethylene glycol monomethyl ether, poly(ethylene glycol), poly(propylene glycol) and glycerol. For example, in some embodiments, the non-aqueous carrier liquid comprises propylene glycol. In another example, in some embodiments, the non-aqueous carrier liquid comprises at least one of ethylene glycol (EG), propylene glycol (PG), poly(ethylene glycol), poly(propylene glycol), diethylene glycol (DEG), ethanol (EtOH) and glycerol (Gly).

The non-aqueous carrier liquid can be selected on the basis of viscosity in order to effectively apply the curable mixture to the tooth. In some embodiments, the non-aqueous carrier liquid has a viscosity (e.g., a bulk viscosity) at 25° C. of about 0.5 cps (centipoise) when measured, for example, on a Brookfield viscometer. In other embodiments, the non-aqueous carrier liquid has a viscosity at 25° C. of about 1 cps, about 2 cps, about 3 cps, about 5 cps, about 10 cps, about 15 cps, about 20 cps, about 23 cps, about 24 cps, about 25 cps or about 30 cps, or any range of values therebetween. The non-aqueous carrier liquid can have a viscosity at 25° C. in the range of about 0.5 cps to about 60 cps, about 0.5 cps to about 40 cps, about 0.5 cps to about 30 cps, about 0.5 cps to about 20 cps, about 20 cps to about 60 cps, about 20 cps to about 40 cps, or about 20 cps to about 30 cps.

Various secondary carrier liquids are suitable for use in the curable mixture of ingredients (for example, as the second carrier liquid in a two-part curable composition). In some embodiments, the secondary carrier liquid is different than the non-aqueous carrier liquid. In some embodiments, the secondary carrier liquid comprises at least one of water, a water-soluble carrier liquid and a water miscible carrier liquid. In some embodiments, the secondary carrier liquid comprises at least one of water, acetic acid, acetone, acetonitrile, 1-butanol, 2-butanone, ethyl acetate, methanol, ethanol, propanol, butanol, dimethyl sulfoxide, dimethylformamide, 1,4-dioxane, methyl isocyanide, pyridine, tetrahydrofuran and a polyol. Examples of polyols include, in some embodiments, ethylene glycol, propylene glycol, triethylene glycol, poly(ethylene glycol), poly(propylene glycol) and glycerol. For example, in some embodiments, the secondary carrier liquid comprises propylene glycol. In another example, in some embodiments, the secondary carrier liquid comprises at least one of water, ethylene glycol (EG), propylene glycol (PG), poly(ethylene glycol), poly(propylene glycol), diethylene glycol (DEG), ethanol (EtOH) and glycerol (Gly). In some embodiments, the secondary carrier liquid comprises water. In some embodiments, the secondary carrier liquid is an aqueous carrier liquid.

The secondary carrier liquid can be selected on the basis of viscosity in order to effectively apply the mixture to the tooth. In some embodiments, the secondary carrier liquid has a viscosity (e.g., a bulk viscosity) at 25° C. of about 0.5 cps, about 1 cps, about 2 cps, about 3 cps, about 5 cps, about 10 cps, about 15 cps, about 20 cps, about 23 cps, about 24 cps, about 25 cps or about 30 cps, or any range of values therebetween. For example, the secondary carrier liquid can have a viscosity at 25° C. in the range of about 2 cps to about 25 cps. In some embodiments, the secondary carrier liquid has a viscosity at 25° C. in the range of about 0.1 cps to about 1000 cps, such as, from about 0.1 cps to about 500 cps, about 0.1 cps to about 100 cps, about 0.1 cps to about 50 cps, about 0.1 cps to about 20 cps, about 0.1 cps to about 15, about 0.1 cps to about 10 cps, and about 0.1 cps to about 1 cps. In some embodiments, the secondary carrier liquid has a viscosity at 25° C. in the range of about 1 cps to about 100 cps, about 1 cps to about 50 cps, about 1 cps to about 40 cps, about 1 cps to about 30 cps, about 1 cps to about 20 cps, or less than 20 cps at 25° C., such as, about 1 cps to about 18 cps, about 1 cps to about 15 cps, or about 1 cps to about 12 cps.

In some embodiments, a first carrier liquid (e.g. the non-aqueous carrier liquid) can be applied to the dry ingredients of the curable mixture to form a paste, and then a second carrier liquid (e.g. the secondary carrier liquid) can be applied to the paste to form the curable mixture. The term “total carrier liquid” refers to the combined first and second carrier liquid of the curable mixture. The use of a first carrier liquid and a second carrier liquid may enable more convenient application of the curable mixture to a tooth, and better control of material characteristics of each part, such as viscosity and setting time. In some embodiments, at least one of the first carrier liquid and the second carrier liquid comprises water. In some embodiments, at least one of the first carrier liquid and the second carrier liquid is substantially anhydrous. In one example, in some embodiments, the curable mixture comprises at least one of water, ethylene glycol (EG), propylene glycol (PG), diethylene glycol (DEG), ethanol (EtOH) and glycerol (Gly). In some embodiments, the first carrier liquid may be selected so that, in combination with calcium silicate and filler, the first part of a two-part curable mixture forms a flowable paste. The first part of the curable mixture may have a flow rate of at least about 20 grams per minute (g/min) at 20 psi when measured, for example, by the Paste Flow Rate test method provided herein. In some embodiments, the first part of the two-part curable mixture has a flow rate in the range of about 20 g/min. to about 1000 g/min., or about 50 g/min. to about 500 g/min., or about 100 g/min. to about 500 g/min., or about 100 g/min. to about 400 g/min., at 20 psi. In some embodiments, the first part of the two-part curable mixture has a flow rate in the range of about 50 g/min. to about 500 g/min. for at least 4 days.

The curable mixture can contain various amounts of the total carrier liquid (which itself may be a mixture). The amount of total carrier liquid can be the balance of the weight of the mixture after the amounts of the other ingredients have been specified. For example, if the total of the amounts of the other ingredients (e.g., calcium silicate, filler material(s), and/or X-ray radiopaque material) is 30 wt. % of the curable mixture, then the amount of the carrier liquid can be the remaining balance, i.e., 70 wt. % of the curable mixture. In some embodiments, the curable mixture comprises an amount of total carrier liquid in any one of the following ranges, such as 45 wt. % to 60 wt. %, 50 wt. % to 80 wt. %, 50 wt. % to 75 wt. %, 50 wt. % to 70 wt. %, 50 wt. % to 65 wt. %, 50 wt. % to 60 wt. %, 55 wt. % to 80 wt. %, 55 wt. % to 75 wt. %, 55 wt. % to 73 wt. %, 55 wt. % to 70 wt. %, 55 wt. % to 65 wt. %, 57 wt. % to 69 wt. % or 60 wt. % to 70 wt. %, total carrier liquid.

In various embodiments, at least a portion of the curing of the curable mixture takes place after positioning the curable mixture in a cavity or root canal. For example, an embodiment provides a method of filling a tooth, comprising identifying a tooth having a cavity in need of filling; positioning a curable mixture as described herein within the cavity; and curing the curable mixture within the cavity. Another embodiment provides a method of filling a tooth, comprising identifying a tooth having a root canal in need of filling; positioning a curable mixture as described herein within the root canal; and curing the curable mixture within the root canal. The positioning of the curable mixture in the cavity or root canal can be carried out in various ways as described elsewhere herein.

In some embodiments, the curable mixture can contain additional components. In some embodiments, the curable mixture can further contain an accelerating agent to accelerate the setting time of the curable mixture. Various accelerating agents can be included in the curable mixture. For examples, in some embodiments, an accelerating agent comprises at least one of calcium chloride, calcium carbonate and calcium sulfate.

In some embodiments, the curable mixture can further contain a surface-active agent to facilitate penetration of the uncured or partially cured curable mixture into small spaces within the tooth and/or root canal system. In an embodiment, the surface-active agent is substantially anhydrous. Various surface-active agents can be included in the curable mixture. For examples, in some embodiments, the surface-active agent comprises at least one of a polysorbate and a sorbitan ester. In some embodiments, the polysorbate is selected from polysorbate 20, polysorbate 40, polysorbate 60, polysorbate 80, and mixtures thereof. In some embodiments, the polysorbate is polysorbate 60. In some embodiments, the sorbitan ester is sorbitan sesquioleate.

The curable mixture can contain various amounts of the surface-active agent. In some embodiments, the curable mixture comprises a surface active agent in any one of the following ranges, such as about 0 wt. % to about 5 wt. %, about 1 wt. % to about 5 wt. %, about 0 wt. % to about 3 wt. %, about 1 wt. % to about 3 wt. %, or about 0.01 wt. % to about 0.1 wt. %.

In some embodiments, the curable mixture can further contain a phosphate salt to facilitate generation of hydroxyapatite. In an embodiment, the phosphate salt is substantially anhydrous. Various phosphate materials can be included in the curable mixture. For examples, in some embodiments, the phosphate salt comprises at least one of calcium hydrogen phosphate and calcium dihydrogen phosphate.

The curable mixture can contain various amounts of the phosphate salt. In some embodiments, the curable mixture comprises an amount of a phosphate salt in any one of the following ranges, such as about 0 wt. % to about 10 wt. %, about 2 wt. % to about 8 wt. %, about 0 wt. % to about 5 wt. %, about 2 wt. % to about 5 wt. %, or about 0.1 wt. % to about 1 wt. %.

In some embodiments, the ingredients (a), (b), (c), and (d) are selected to provide the curable mixture with a viscosity effective to permit flow into the complex anatomy of a tooth. In some embodiments, the tooth comprises a filling space with a diameter of about 150 μm to about 200 μm. In some embodiments, the tooth comprises a filling space with a diameter in the range of about 150 μm to about 200 μm at the base. In some embodiments, the ingredients (a), (b), (c), and (d) are selected to provide the curable mixture with properties suitable for use as a root canal filling after curing by the exposure of the curable mixture to the effective amount of water.

In some embodiments, the curable mixture comprises one or more ingredients that can be expressed through an opening or an orifice of less than 100 um. In various embodiments, the ingredients and the final curable mixture are stable and can function in temperatures minimally between 0° C. and 50° C.

In some embodiments, the curable mixture has a viscosity that facilitates delivery of the curable mixture into a filling space in the tooth at a temperature of 37° C., the filling space having a diameter in the range of about 150 μm to about 200 μm at an apex of the filling space. In some embodiments, the curable mixture comprises one or more materials that can be expressed through an opening of less than 100 um.

### Hydraulic Cement Mixture

In some embodiments, the curable mixture is a curable hydraulic cement mixture that comprises about 5-20 wt. % tricalcium silicate; 0.1-1.5 wt. % fumed silica; 20-30 wt. % YbF₃; and the balance comprising at least one carrier liquid (such as a polyol), water and, optionally, a secondary X-ray radiopaque material, a hygroscopic material, an accelerating agent, a surface active agent and/or a retardant. In some such embodiments, the water is introduced in the second part of a two-part curable mixture.

In some aspects, the present disclosure describes an advanced curable mixture comprising a calcium silicate compound. The various ingredients of the mixture are selected to provide a curable mixture. In various embodiments, the obturation material mixture is cured by exposure to water and thus the uncured or curable mixture can be substantially anhydrous in order to prolong shelf and operatory working time. In this context, the term “substantially anhydrous” refers to an uncured obturation material mixture that, in the absence of moisture, does not exhibit curing for a period of at least 12 hours. For example, in various embodiments the substantially anhydrous curable mixture contains less than 1 wt. % water, less than 0.5 wt. % water or less than 0.1 wt. % water. In various other embodiments, water is added to form the obturation material mixture and thus initiate curing of the curable mixture.

Various beneficial characteristics of embodiments of the curable mixture are described herein. In some embodiments, the ingredients of the mixture are selected to provide properties suitable for use as a tooth filling after curing by exposure of the mixture to an effective amount of water. Curing of the curable mixture by exposure to water can enable the curable mixture to be cured after it is applied to the tooth, as moisture inside a patient's own mouth can be used to cure the mixture. Additionally, an external water source can also be used to cure or to assist cure of the curable mixture. For example, in some embodiments, water can be added to form the uncured obturation material mixture before or as the mixture is applied to the tooth (for example, by mixing the first part and the second part of a two-part curable mixture, where water is present in the second part).

In some embodiments, the curable mixture has a viscosity that is effective to permit flow into the complex anatomy of a tooth. For example, in some embodiments, the curable mixture has a viscosity effective to permit flow into the complex anatomy of a tooth, wherein the tooth comprises a filling space with a diameter as described elsewhere herein. For example, in some embodiments, the filling space diameter is a cross-sectional dimension at the apex of the space, as described elsewhere herein. In another example, in some embodiments, the filling space diameter is a cross-sectional dimension at a coronal portion of the space, as described elsewhere herein. In some embodiments, the curable mixture is suitable for use as a root canal filling after curing by exposure of the curable mixture to an effective amount of water.

In various embodiments, the curable mixture comprises effective amounts of a carrier liquid that is substantially water free, a calcium silicate compound, a filler material, and an X-ray radiopaque material, as described in greater detail herein. In various other embodiments, the curable mixture comprises effective amounts of a first carrier liquid that is substantially water free, a second carrier liquid comprising >1% water, a calcium silicate compound, a filler material, and an X-ray radiopaque material, as described in greater detail herein.

In some embodiments, the curable mixture can be hardened by mixing with water (e.g. from the secondary carrier liquid) or due to moisture inside a tooth, a root canal system, or a treatment region. In some embodiments, a mixture can be hardened or cured without the need for an external energy source. In some embodiments, a mixture can be hardened or cured without the need for an additional curing agent.

In various embodiments obturation materials for use as tooth fillings are formed from a curable mixture of ingredients that, when cured or during a subsequent cure phase, have one or more of several desirable properties. For example, in some embodiments, the obturation material is biocompatible. In some embodiments, the obturation material is x-ray radiopaque. In some embodiments, the curable mixture of ingredients further provides the obturation material with dimensional stability after cure. In some embodiments, the curable mixture of ingredients has minimal or no shrinkage upon setting. In some embodiments, the curable or cured material is readily removed if necessary.

### Methods of Use

The curable mixtures described herein can be used in various methods of filling a tooth. In some embodiments, the method comprises positioning the curable mixture within a cavity. In some embodiments, the method comprises exposing the curable mixture (e.g., a substantially anhydrous curable mixture) within the cavity to water for a period of time effective to cure the mixture.

In some embodiments, the curable mixture can be used in a method of filling a root canal. In some embodiments, the method comprises positioning the curable mixture within the root canal. In some embodiments, the method comprises exposing a curable mixture (e.g., a substantially anhydrous curable mixture) within the root canal to water for a period of time effective to cure the mixture. In some embodiments, the water is from a carrier liquid, bodily fluids, or both. In some embodiments, the first part and second part of a two-part curable mixture is combined as the curable mixture is introduced into the root canal.

In some embodiments, the method comprises curing the curable mixture within the cavity without the need for an external energy source or additional curing agent. In some embodiments, the curable mixture is positioned within the root canal by mixing with a carrier liquid that has a bulk viscosity of about 5 cps, about 10 cps, about 20 cps, about 30 cps, about 50 cps, about 75 cps, about 100 cps, about 125 cps, about 150 cps, about 170 cps, about 190 cps, about 200 cps or about 250 cps, or any range of values therebetween. For example, in an embodiment, the carrier liquid can have a bulk viscosity in the range of about 10 cps to about 200 cps.

Curable materials described herein can be formed and applied to a tooth by various methods and devices. The obturation material can be formed in any suitable manner. For example, in some embodiments, a clinician can form the obturation material by mixing the obturation material ingredients, e.g., by hand, by a mechanical tool, or by a mixing device. Furthermore, the obturation material can be applied to a tooth in any suitable manner. For example, in some embodiments, a clinician can apply the obturation material by placing it in the tooth, e.g., by hand, by syringe, by a mechanical tool, or by an application device, such as a device as described in U.S. Pat. No. 9,877,801.

In some embodiments, the curable mixture has a viscosity that facilitates delivery of the obturation material into a cavity or space in need of repair. In some embodiments the cavity is a root canal. In some embodiments, the curable mixture has a viscosity that facilitates delivery of the obturation material to a cavity without requiring the use of an obturation point or other mechanical means to deliver the curable mixture to the base of the cavity. In some embodiments, the cavity has a first cross-sectional dimension at the apex of the filling space of about 100 μm, about 125 μm, about 150 μm, about 175 μm, about 200 μm, about 225 μm or about 250 μm, or any range of values therebetween. For example, in an embodiment the cavity has a first cross-sectional dimension at the apex of the cavity in the range of about 150 μm to about 200 μm. In some embodiments, the cavity has a second cross-sectional dimension at a coronal portion of the filling space of about 500 μm, about 600 μm, about 700 μm, about 800 μm, about 900 μm, about 1 mm or about 1.2 mm, or any range of values therebetween. For example, in an embodiment the filling space has a second cross-sectional dimension at a coronal portion of the filling space in the range of about 100 μm to about 4 mm.

In some embodiments, the curable mixture of ingredients comprises one or more parts. In a “one-part” embodiment, the ingredients of the curable mixture are combined together in a single composition. In a “two-part” embodiment, one or more ingredients of the mixture are contained in a first part, one or more ingredients are contained in a second part, and curing commences at a time after the first and second parts are combined. Similarly, in a “three-part” embodiment, one or more ingredients of the mixture are contained in a first part, one or more ingredients are contained in a second part, one or more ingredients of the mixture are contained in a third part, and curing commences at a time after the first, second and third parts are combined. A curable mixture as described herein will thus be understood to be in the form of one, two, three or more parts, unless the context indicates otherwise. In some embodiments, the one or more parts of the mixture can be expressed through an opening of less than, or about, 10 μm, 50 μm, 60 μm, 80 μm, 100 μm, 150 μm, 200 μm, or any range of values therebetween. For example, in an embodiment one or more parts can each individually be expressed through an opening in the range of about 50 μm to about 150 μm, or less than 100 μm. In some embodiments, two are more parts of the curable mixture are combined as the mixture is introduced into a cavity or space in need of repair.

### Application Device

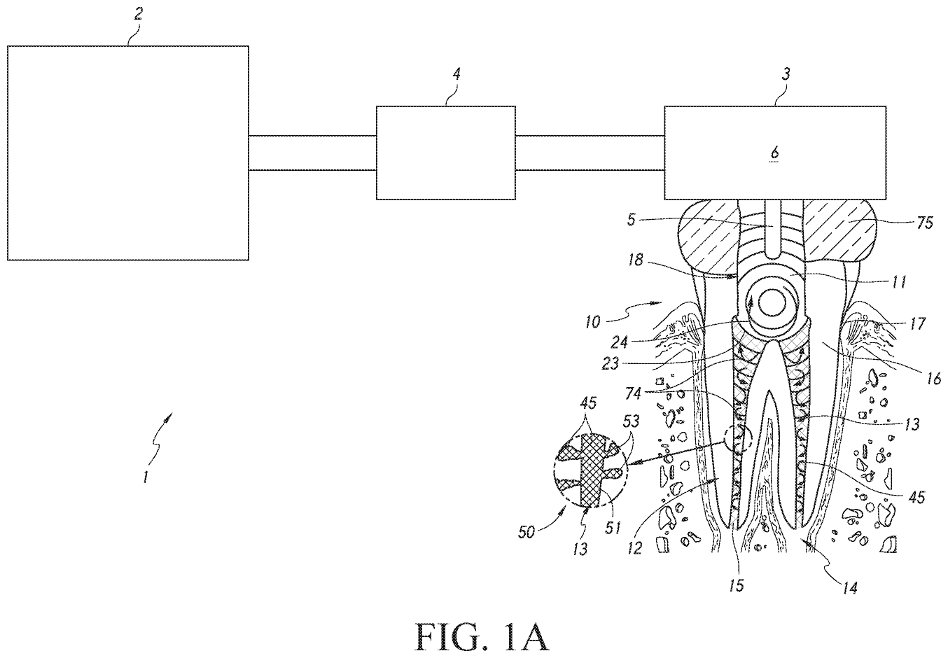

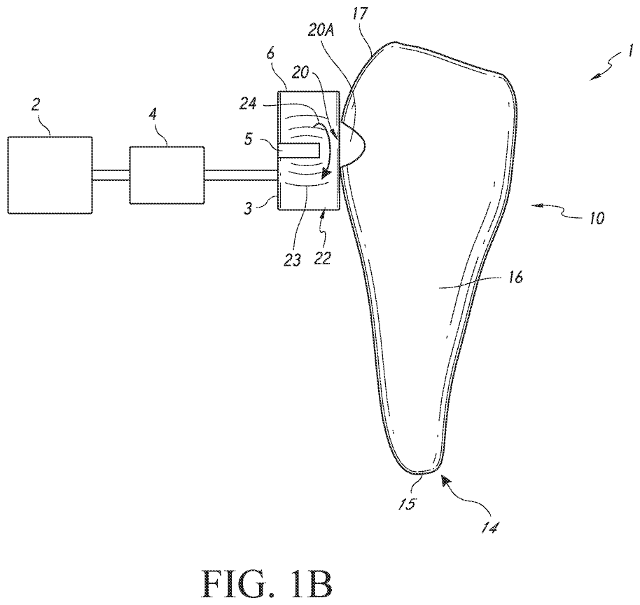

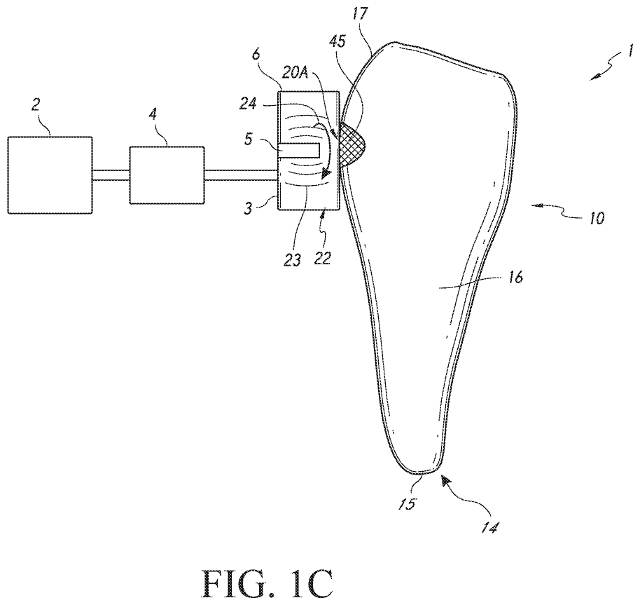

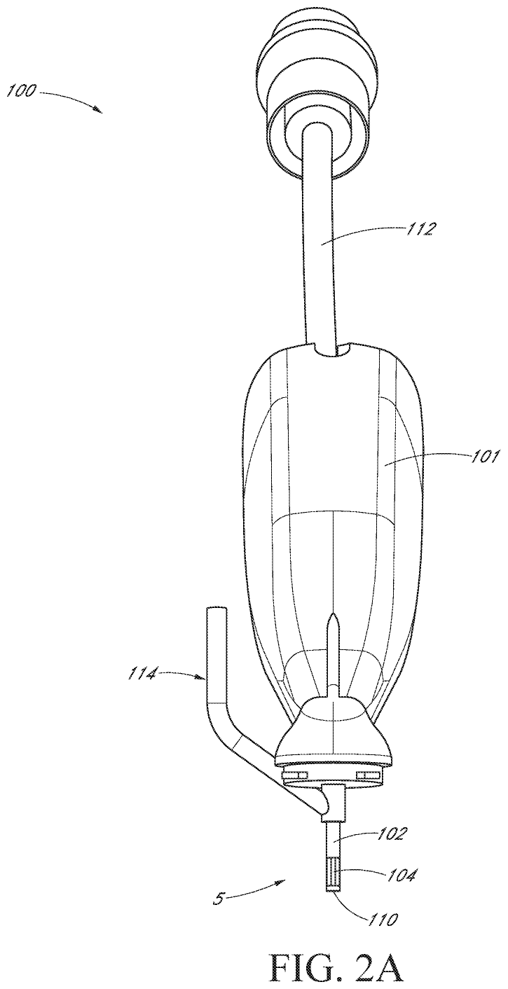

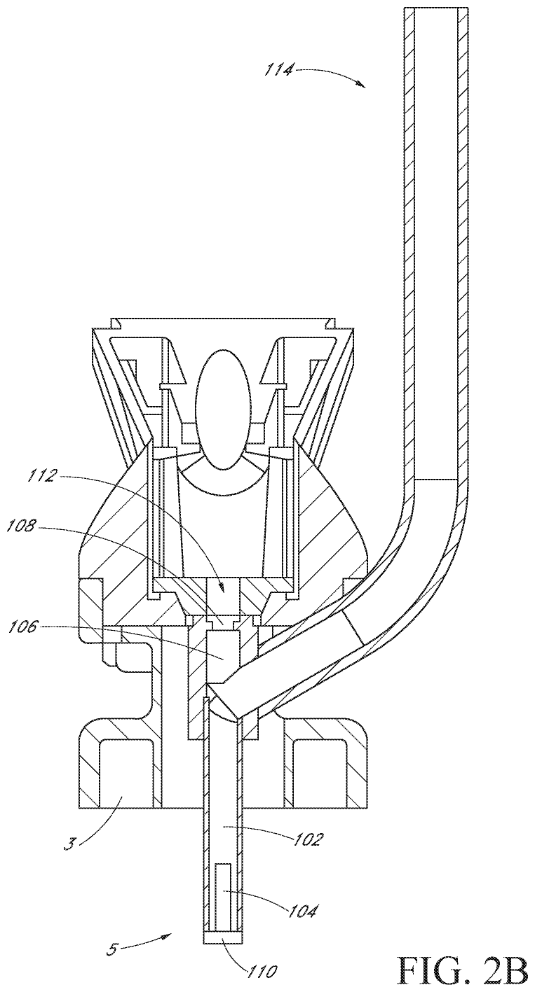

The curable materials and obturation materials described herein can be formed and applied to a tooth by various methods and devices. The filling or obturation material can be formed in any suitable manner. For example, in some embodiments, a clinician can form the obturation material by mixing the obturation material ingredients, e.g., by hand, by a mechanical tool, or by a mixing device. Furthermore, the obturation material can be applied to a tooth in any suitable manner. For example, in some embodiments, a clinician can apply the obturation material by placing it in the tooth, e.g., by hand, by syringe, by a mechanical tool, or by an application device. As described below and in FIGS. 1-2B, embodiments of a mixing device and/or an application device that can be used to form and/or apply an obturation material are disclosed.

FIG. 1A is a schematic diagram of a system 1, in accordance with embodiments of an application or delivery device as disclosed herein. The system 1 can be configured to perform various types of treatment procedures, including, e.g., cleaning treatments, obturation or other filling treatments, restoration treatments, etc. In the embodiment shown in FIG. 1A, the system 1 is illustrated as being coupled to (e.g., positioned against in some arrangements) a tooth 10 that is a molar tooth of a mammal, such as a human. However, the tooth 10 can be any other suitable type of tooth, such as a pre-molar, bicuspid, incisor, canine, etc. Furthermore, the system 1 shown in FIG. 1A can include components configured to remove unhealthy or undesirable materials from a tooth or surrounding gum tissue, for example, a root canal 13 of the tooth 10. Thus, in the embodiment of FIG. 1A, the system 10 can also be configured to clean the tooth 10, in addition to being configured to fill or obturate the tooth. Moreover, although the treatment shown in FIG. 1A is a root canal treatment, in other embodiments, the application device and obturation material(s) disclosed herein can be used to fill other types of treatment regions, such as a treated carious region of the tooth.

The tooth 10 includes hard structural and protective layers, including a hard layer of dentin 16 and a very hard outer layer of enamel 17. A pulp cavity 11 is defined within the dentin 16. The pulp cavity 11 comprises one or more root canals 13 extending toward an apex 14 of each root 12. The pulp cavity 11 and root canal 13 contain dental pulp, which is a soft, vascular tissue comprising nerves, blood vessels, connective tissue, odontoblasts, and other tissue and cellular components. Blood vessels and nerves enter/exit the root canal 13 through a tiny opening, the apical foramen or apical opening 15, near a tip of the apex 14 of the root 12. It should be appreciated that, although the tooth 10 illustrated herein is a molar, the embodiments disclosed herein can advantageously be used to treat any suitable type of tooth, including pre-molars, canines, incisors, etc.

The system 1 can include a console 2, a pressure wave generator 5, and a tooth coupler 3 (such as a handpiece) adapted to couple to the tooth 10. The tooth coupler 3 can couple to the tooth 10 in any suitable way. In some arrangements, the tooth coupler 3 can be positioned against and/or attach to the tooth 10 by way of a tooth seal 75. For example, the clinician can hold the tooth coupler 3 against the tooth 10 during treatment. In some embodiments, the tooth coupler 3 can define a chamber 6 configured to retain fluid therein, such as a filler or obturation material described herein. In some embodiments, the pulp cavity 11 can define a tooth chamber configured to retain fluid therein. In some embodiments, the tooth coupler 3 may not define a chamber, and the tooth chamber defined at least in part by the pulp cavity 11 can retain fluid. The tooth coupler 3 disclosed herein can be any suitable structure or housing configured to couple to the tooth 10 for a treatment procedure. As used herein, “couple” is meant to include arrangements in which there is a connection with the tooth 10, as well as arrangements in which the coupler 3 is placed against or in the tooth and is held by the clinician in that position. The pressure wave generator 5 can be coupled to and/or disposed in or on the tooth coupler 3 in various embodiments.

A system interface member 4 can electrically, mechanically, and/or fluidly connect the console 2 with the tooth coupler 3 and pressure wave generator 5. For example, in some embodiments, the system interface member 4 can removably couple the tooth coupler 3 to the console 2. In such embodiments, the clinician can use the tooth coupler 3 one time (or a few times), and can dispose the tooth coupler 3 after each procedure (or after a set number of procedures). The console 2 and interface member 4 can be reused multiple times to removably couple (e.g., to connect and/or disconnect) to multiple tooth couplers 3 using suitable engagement features, as discussed herein. The interface member 4 can include various electrical and/or fluidic pathways to provide electrical, electronic, and/or fluidic communication between the console 2 and the tooth coupler 3. The console 2 can include a control system and various fluid and/or electrical systems configured to operate the pressure wave generator 5 during a treatment procedure. The console 2 can also include a management module configured to manage data regarding the treatment procedure. The console 2 can include a communications module configured to communicate with external entities about the treatment procedures. Additionally, the console 2 can include a control system comprising a processor and non-transitory memory. Computer-implemented instructions can be stored on the memory and can be executed by the processor to assist in controlling cleaning and/or filling procedures. Additional details of the console 2 can be found in U.S. Pat. No. 9,504,536, and in U.S. Pat. No. 9,675,426, each of which is incorporated by reference herein in its entirety and for all purposes.

In FIG. 1A, the system 1 is used to fill or obturate the root canal 13 with an obturation material 45, which can be the same as or generally similar to the filler materials described herein. For example, the clinician can clean the root canal 13 in any suitable way, such as by using drills or files, or by using a pressure wave generator (which can be the same as or different from the pressure wave generator 5 shown in FIG. 1A). When the root canal 13 is cleaned, the clinician can supply an obturation material 45 in its flowable state to the pulp cavity 11, canals 13, or other internal chambers of the tooth 10.

As explained herein, the clinician can supply the obturation material 45 to the treatment region (e.g., the root canal) in any suitable manner. For example, in some embodiments, the pressure wave generator 5 (which can be coupled to or formed with a handpiece) can have one or more openings configured to deliver the flowable obturation material 45 to the tooth 10. In other embodiments, the clinician can supply the obturation material 45 to the tooth by manually placing it in the tooth 10, e.g., by hand, by syringe, or by a mechanical tool. In still other embodiments, a dental handpiece can include one or more supply lines that are configured to route the flowable obturation material 45 to the tooth 10. The obturation material 45 can be any suitable obturation material disclosed herein. In particular, the obturation material 45 can have a flowable state in which the obturation material 45 flows through the treatment region to fill the root canals 13 and/or pulp cavity 11. The obturation material 45 can have a hardened state in which the obturation material 45 solidifies after filling the treatment region. In some embodiments, flowable obturation material is supplied by providing a flowable two-part curable mixture comprising a first flowable part and a second flowable part. The first and second flowable parts may be combined to create a flowable, curable mixture where curing is initiated upon mixing of the two parts.

Advantageously, the pressure wave generator 5 can be activated to enhance the obturation or filling procedure. For example, the pressure wave generator 5 can be activated to assist in flowing the obturation material 45 throughout the treatment region to be filled. The pressure wave generator 45 can thereby assist in substantially filling the tooth 10. As shown in inset 50 of FIG. 1A, for example, when activated, the pressure wave generator 5 can cause the obturation material 45 to flow into major canal spaces 51 of the tooth 10, as well as into small spaces 53 of the tooth 10. Thus, the system 1 shown in FIG. 1A can assist in filling even small cracks, tubules, and other tiny spaces (e.g., the small spaces 53) of the tooth 10. By filling the small spaces 53 of the tooth, the system 1 can ensure a more robust obturation procedure which results in long-term health benefits for the patient. As explained herein, the pressure waves 23 and/or fluid motion 24 (which can include vortices 74) generated by the pressure wave generator 5 can interact with the obturation material 45 to assist in filling the small spaces 53 and the major spaces 51 of the tooth 10. Furthermore, in some embodiments, the pressure wave generator 5 can be activated to assist in curing or hardening the obturation material 45. For example, as explained herein, some types of obturation materials can cure or harden (or the curing or hardening can be enhanced) when agitated by pressure waves 23 generated by the pressure wave generator 5. In addition, in various embodiments, the obturation or filling material can be degassed, which can help deliver the obturation material to small spaces of the tooth. Accordingly, the pressure wave generator 5 can enhance the obturation procedure in a variety of ways.

In some embodiments, the obturation material 45 is supplied to the tooth 10, and the pressure wave generator 5 is subsequently activated to enhance the obturation procedure (e.g., to improve the filling process and/or to enhance or activate the curing process). For example, in such embodiments, the clinician can supply the obturation material 45 to the tooth 10 using a syringe or other device, and the pressure wave generator 5 can subsequently (or concurrently) be activated to fill the treatment region. In other embodiments, the pressure wave generator 5 can supply the obturation material 45 and generate pressure waves through the obturation material (or other fluids at the treatment region). In some embodiments, supplying the obturation material and generating pressure waves can occur substantially simultaneously, or can overlap by some amount over time. For example, the pressure wave generator 5 can be activated to supply the obturation material 45 to the treatment region. For example, in embodiments in which the pressure wave generator 5 comprises a liquid jet, a jet of obturation material 45 can interact with fluids in the tooth 10 (e.g., other portions of the obturation material or other treatment fluid) to generate pressure waves that propagates through the fluids. The resulting pressure waves can enhance the obturation procedure. In some embodiments using a two-part curable mixture, one part of the two-part mixture may be provided by the pressure wave generator as a liquid jet and a separate flowable part of the mixture may be provided to mix with the liquid jet. In various embodiments, the pressure waves can have a broadband of multiple frequencies, which can further enhance the filling of the treatment region. Additional details regarding the generation of broadband pressure waves is shown and described at least in FIGS. 2A-2C, and the associated disclosure, of U.S. Pat. No. 9,877,801, the entire contents of which are incorporated by reference in their entirety and for all purposes. In other embodiments, different types of fluids (e.g., water or other treatment fluids) can form the jet, and the jet can pass through obturation materials in the treatment region. Interaction of the fluid jet and the obturation material can enhance the obturation procedure.

As disclosed herein, the pressure wave generator 5 can comprise any suitable type of pressure wave generator, e.g., a liquid jet device, a laser, a mechanical stirrer, an ultrasonic transducer, and the like. The pressure wave generator 5 can be sized such that the pressure wave generator 5 is disposed outside the region of the tooth 10 that is to be obturated. For example, the pressure wave generator 5 can be disposed in the chamber 6 such that it is disposed outside the tooth 10. In other arrangements, the pressure wave generator 5 can extend partially into the tooth 10. In some arrangements, the pressure wave generator 5 can extend to a depth that does not interfere with the filling. The system 1 can include a cleaning mode for cleaning the treatment region and a filling mode to fill or obturate the treatment region.

The console 2 can include a control system comprising a processor and memory. The control system can be programmed or configured to switch the system 1 from the cleaning mode to the filling mode and vice versa. The control system of the console 2 can also control the operation of cleaning and/or filling procedures. Additional details of the delivery device shown in FIG. 1A can be found throughout U.S. Pat. No. 9,877,801, the entire contents of which are incorporated herein by reference and particularly for the purpose of describing such details.

FIG. 1B is a schematic diagram of a system 1 that includes components configured to clean unhealthy or undesirable material from a treatment region 20 on an exterior surface of the tooth 10. For example, as in FIG. 1A, the system 1 can include a tooth coupler 3 and a pressure wave generator 5. The tooth coupler 3 can communicate with a console 2 by way a system interface member 4. Unlike the system 1 of FIG. 1A, however, the tooth coupler 3 is coupled to (e.g., positioned against by a clinician) a treatment region 20 on an exterior surface of the tooth 10. In some embodiments, the tooth coupler 3 can be stably positioned against the treatment region and can be sealed to the tooth 10, e.g., by way of an adhesive or other seal. The system 1 of FIG. 1B can be activated to clean an exterior surface of the tooth 10, e.g., a carious region of the tooth 10 and/or remove undesirable dental deposits, such as plaque, calculus biofilms, bacteria, etc, from the tooth 10 and/or surround gum tissue. In other embodiments (see FIG. 1C), the system 1 can be activated to fill a treated region on the exterior surface of the tooth 10 with a filling or restoration material. As with the embodiment of FIG. 1A, pressure waves 23 and/or fluid motion 24 can be generated in the tooth coupler 3 and chamber 6, which can act to clean the treatment region 20 of the tooth 10, forming a cleaned treatment region 20A in which the carious (or other unhealthy material) is removed. Additional details of systems and methods for treating carious regions of teeth can be found in International Application Publication WO 2013/142385 (PCT/US2013/032635), having an international filing date of Mar. 15, 2013, entitled “APPARATUS AND METHODS FOR CLEANING TEETH,” the entire contents of which are incorporated by reference herein in their entirety and for all purposes. Additional details of systems and methods for removing undesirable dental deposits (such as plaque, calculus, etc.) from teeth and/or gums can be found in International Application Publication WO 2013/155492 (Application No. PCT/US2013/036493), having an international filing date of Apr. 12, 2013, entitled “APPARATUS AND METHODS FOR CLEANING TEETH AND GINGIVAL POCKETS,” and in U.S. Patent Publication No. US 2014/0099597, filed Apr. 11, 2013, entitled “APPARATUS AND METHODS FOR CLEANING TEETH AND GINGIVAL POCKETS,” each of which is incorporated by reference herein in its entirety and for all purposes.

FIG. 1C is a schematic diagram of the system 1 of FIG. 1B, in which the system 1 is configured to fill the treated carious region 20A of the tooth 10, and can be used in combination with any of the filling materials disclosed herein. As with the embodiment of FIG. 1B, the system can include a pressure wave generator 5, a tooth coupler 3, an interface member 4, and a console 2. When the carious or other unhealthy material is removed from the tooth 10, the clinician can fill the cleaned treatment region 20A with a suitable filler or obturation material 45. As with the embodiment of FIG. 1A, the obturation material 45 can be supplied to the cleaned treatment region 20A. The pressure wave generator 5 can act to substantially fill the treatment region 20A and/or to enhance or activate the hardening of the filler obturation material 45. In some embodiments, the filler or obturation material 45 is supplied to the tooth 10, and the pressure wave generator 5 is subsequently activated to enhance the filling procedure (e.g., to improve the filling process and/or to enhance or activate the curing process). For example, in such embodiments, the clinician can supply the filler or obturation material 45 to the treatment region 20A using a syringe, and the pressure wave generator 5 can subsequently be activated to fill the treatment region. In other embodiments, the pressure wave generator 5 is activated to supply the filler or obturation material 45 to the treatment region 20A and to generate pressure waves through the material. For example, in embodiments in which the pressure wave generator 5 comprises a liquid jet, a jet of obturation or filler material 45 (or other type of fluid) can interact with fluids at the treatment region 20A (e.g., other portions of the filler or obturation material or other treatment fluid) to generate pressure waves that propagates through the fluids. The resulting pressure waves can enhance the obturation procedure.

FIGS. 2A and 2B depict a delivery device 100 that can be used to combine a first composition with a second composition to form the curable mixture and apply it to a treatment region of the tooth to fill the treatment region. As shown in FIGS. 2A-2B, the delivery device 100 can comprise a treatment instrument 101. The treatment instrument 101 can be used to position the pressure wave generator 5 at or near the treatment region. In the embodiment of FIG. 2A, the treatment instrument 101 comprises a handpiece sized and shaped to be held by the clinician against a portion of the tooth. Further, the delivery device 100 can comprise a first composition supply line 112 and a second composition supply line 114. The first composition supply line 112 can be configured to supply the first composition to a distal portion of the handpiece 101. The second composition supply line 114 can be configured to supply the second composition to the distal portion of the handpiece 101. For example, in some embodiments, the first composition supply line 112 can be configured to supply the carrier liquid to the tooth, and the second composition supply line 114 can supply other component materials to mix with the carrier liquid.

In FIG. 2A, a pressure wave generator 5 can be coupled to or formed with the distal portion of the handpiece 101. As explained above in connection with FIGS. 1A-1C, the pressure wave generator 5 can be activated to generate pressure waves and/or fluid motion at the treatment region, to cause the filling or obturation material to fill the treatment region. As explained above, the pressure wave generator 5 can comprise any suitable type of pressure wave generator, including those described in U.S. Pat. No. 9,877,801, the entire contents of which are incorporated herein by reference in their entirety and for all purposes. For example, the pressure wave generator 5 of FIGS. 2A-2B comprises a liquid jet device. The liquid jet device can comprise a nozzle or orifice 108 sized and shaped to pressurize a composition that is supplied to the orifice 108 by way of the first composition supply line 112. In some embodiments, the orifice 108 can form a composition into a liquid jet, e.g., a coherent, collimated liquid jet. The liquid jet formed of a composition can pass into a mixing chamber 106 disposed distal the orifice 108. Thus, in FIG. 2B, the second supply line 114 can be positioned so as to deliver another composition to the mixing chamber 106 at a location distal the orifice 108. Thus, the liquid jet of, for example, the carrier liquid, can be formed and can pass through the mixing chamber 106 to interact with other component materials supplied by the second supply line 114. In one embodiment, the first part of a two-part curable mixture is supplied through the second supply line 114 and the second part of the two-part curable mixture is supplied through the first composition supply line 112. For example, a calcium silicate paste composition comprising a non-aqueous carrier liquid, calcium silicate and filler, may be supplied through the second supply line 114, and a second carrier liquid (e.g., a low viscosity aqueous liquid carrier) may be supplied through the first supply line 112.

As shown in FIG. 2B, the second supply line 114 can supply the first part of the curable mixture composition to the mixing chamber 106 by way of one or more ports. The first and second parts of the curable mixture composition can accordingly be mixed within the mixing chamber 106 to at least partially form the mixed composition (e.g., curable mixture) of the filling or obturation material. The momentum of the liquid jet can drive the at least partially mixed parts of the curable mixture composition along a guide tube 102. The liquid jet can impinge on an impingement member 110 located at a distal portion of the guide tube 102. The delivery device 100 can comprise a side port delivery device in which the curable mixture is supplied to the device (e.g., under 20 psi pressure) to the treatment region through one or a plurality of openings 104 in the guide tube 102. The openings 104 can be disposed proximal the impingement member 110. Interaction of the at least partially mixed first and second parts of the curable mixture composition with fluid in the treatment region can generate pressure waves and/or fluid motion at the treatment region. The pressure waves and/or fluid motion can assist in filling or obturating the treatment region. Additional details of liquid jet devices used for filling a treatment region can be found in FIGS. 4A-8D of U.S. Pat. No. 9,877,801, the entire contents of which are incorporated by reference herein in their entirety and for all purposes.

Accordingly, in some embodiments, first and second parts of the curable mixture composition can be kept separate until combined in the mixing chamber 106 of the delivery device 100 to form the curable mixture. In some embodiments, combination of the second part decreases the viscosity of the first part in order to create a curable mixture suitable for delivery to the treatment region. In some embodiments, curing or hardening of the curable obturation material is initiated when combined. In some embodiments, at least one of the first and second parts are introduced into the curable mixture as a fluid jet as explained herein.

Although the examples shown in FIGS. 1-2B describe the delivery device as including a pressure wave generator, it should be appreciated that the obturation material(s) described herein can be used in conjunction with any other suitable type of delivery device. For example, the obturation material(s) described herein can be used with a syringe, a mechanical instrument, or any other suitable device.

### Test Methods

### Radiopacity Measurement

The radiopacity of cured compositions formed from the curable mixtures was measured by reference to a specimen of an aluminum (Al) standard according to ISO 6876:2012.

### Paste Flow Rate

The flow rate of the first part of the curable mixture was tested according to the following method. A Nordsen EFD (Ultimus 1; part number 7017041) was set to 20 psi and a timer was set for 3 to 5 seconds. A female luer was paired to ⅛″ barb fitting, and the barb was lightly coated with silicon. A 1.7″ length of Tygon S3™ tubing was placed over a ⅛ barb, and fitting was attached to a syringe loaded with about 10 ml to 20 ml of test sample paste. Paste was extruded into the Tygon tubing until flush with the tubing end, and the syringe was connected to the EFD adapter at ambient temperature. A weigh boat was placed on scale and tared. The syringe was held perpendicular to the weigh boat and the paste was extruded for 3 seconds to 5 seconds. The weight of the extruded paste was noted, and the flow rate was reported in grams/minute (g/min.).

### EXAMPLES

### Example 1: Preparation of Hydraulic Cement Mixture

Two-part curable material compositions were prepared. The ingredients of each curable mixture formulation comprising a tricalcium silicate compound are listed as Examples 1 through 8 in Table 1 below. Material compositions made according to Examples 1 through 8 were each formed in two flowable parts that were combined to form a curable mixture. The curable mixtures were suitable to function as dental obturation materials when cured, for example, for use as a root canal filling material.

TABLE 1
Two-Part Curable Material Compositions.

| Ingredients | | | | | | | | | |
|---|---|---|---|---|---|---|---|---|---|
| (wt. %) | Ex. 1 | Ex. 2 | Ex. 3a | Ex. 3b | Ex. 4 | Ex. 5 | Ex. 6 | Ex. 7 | Ex. 8 |
| Fumed | 1% | 1% | — | — | 0.5% | 0.5% | — | 1% | 0.94% |
| Silica | | | | | | | | | |
| Tricalcium | 8.5% | 8.5% | 15% | 15% | 15% | 15% | 15% | 15% | 14.06% |
| Silicate | | | | | | | | | |
| Ytterbium | 25.5% | 28% | 25% | 25% | 24.5% | 24.5% | 25% | 23.5% | 22.04% |
| Fluoride | | | | | | | | | |
| PEG-300 | 15% | 12.5% | — | — | — | — | — | — | — |
| Propylene | — | — | 10% | 10% | 10% | 10% | 7.5% | 10% | 12.5% |
| Glycol | | | | | | | | | |
| Potassium | — | — | — | — | — | — | 2.5% | — | — |
| Iodide (KI) | | | | | | | | | |
| Magnesium | — | — | — | — | — | — | — | 0.5% | 0.46% |
| Sulfate | | | | | | | | | |
| Flow Rate | — | — | — | — | T0: 212.5 | — | — | T0: 158.9 | — |
| @20 psi | | | | | T4: 190.8 | | | T3: 135.8 | |
| (g/min) of | | | | | T6: 156.9 | | | T8: 146.6 | |
| First Part | | | | | | | | T10: 157.8 | |
| Water | 50% | 50% | 50% | 50%* | 50% | 40% | 50% | 50% | 50% |
| | | | | (w KI) | | | | | |
| Calcium | — | — | — | — | — | 10% | — | — | — |
| Chloride | | | | | | | | | |
| Radiopacity | — | — | 2 | 9* | 2 | 2 | 2 | 2 | 2 |
| (mmA1) | | | | | | | | | |

*Part 2 was prepared as a 1:1 weight ratio water: 55 wt % KI aqueous solution)

For each example, a first part was prepared by mixing a filler (i.e., fumed silica), radiopaque agent (i.e., ytterbium fluoride), tricalcium silicate, and a non-aqueous liquid carrier (e.g., PEG-300 or propylene glycol). Flow rates of the first parts of Example 4 and Example 7 were measured according to the method provided herein for Paste Flow Rate. Measurements were taken over several days to confirm stability, and the results are reported in Table 1. The first part of each of Examples 4 and 7 had an initial flow rate (TO) at 20 psi and ambient temperature of 212.5 grams per minute (g/min) and 158.9 g/min, respectively, and maintained an acceptable flow rate of 156.9 g/min (day 6) and 157.8 (day 10), for Exs. 4 and 7, respectively.

The second parts of each example comprised an aqueous carrier liquid, and optionally, magnesium sulfate, calcium chloride, and/or potassium iodide (as a radiopaque agent). After mixing the first and second parts, all curable compositions formed silicate cements. Radiopacity measurements were obtained for cured compositions of Examples 3 through 8. Example 3 having a first part comprising a first radiopaque material, and a second part (i.e., water) having a second radiopaque material (potassium iodide (KI)), demonstrated a higher radiopacity (radiopacity 9) than Examples 4 through 8 (radiopacity=2), having a radiopaque material in only the first component. Examples were tested for robustness by drying overnight at ambient conditions, and then rinsing under a water stream. Examples 1 and 2 rinsed off of the laboratory substrate, and were therefore deemed less robust than Examples 3 through 8, which remained intact.

The curable mixtures of Exs. 1 through 8 may be suitable for delivery to a tooth space by a delivery device, such as the devices shown in FIGS. 1 through 2B. For example, the flow rate of the first part of each example was suitable for delivery through a side port at 20 psi, and the viscosity of the second part (e.g., below 20 cps measured on a Brookfield viscometer at about 25° C.) was sufficiently low to form a liquid jet within the device, and to form a homogenous mixture with the first part, within the device.

While certain embodiments have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount, depending on the desired function or desired result.

The headings contained in this document, if any, are for convenience only and do not necessarily affect the scope or meaning of the devices and methods disclosed herein.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A curable mixture of ingredients, comprising:
(a) a calcium silicate compound;
(b) a filler material;
(c) a non-aqueous carrier liquid; and
(d) a secondary carrier liquid different than the non-aqueous carrier liquid.

2. The curable mixture of claim 1, wherein the calcium silicate compound is selected from at least one of calcium silicate, dicalcium silicate and tricalcium silicate.

3. The curable mixture of claim 1, wherein the non-aqueous carrier liquid comprises at least one of acetic acid, acetone, acetonitrile, 1-butanol, 2-butanone, ethyl acetate, methanol, ethanol, propanol, butanol, dimethyl sulfoxide, dimethylformamide, 1,4-dioxane, methyl isocyanide, pyridine, tetrahydrofuran, ethylene glycol, propylene glycol, triethylene glycol, poly(ethylene glycol), poly(propylene glycol) and glycerol.

4. (canceled)

5. The curable mixture of claim 1, wherein the secondary carrier liquid comprises at least one of water, acetic acid, acetone, acetonitrile, 1-butanol, 2-butanone, ethyl acetate, methanol, ethanol, propanol, butanol, dimethyl sulfoxide, dimethylformamide, 1,4-dioxane, methyl isocyanide, pyridine and tetrahydrofuran.

6. (canceled)

7. The curable mixture of claim 1, further comprising an X-ray radiopaque material.

8. The curable mixture of claim 1, further comprising a phosphate salt.

9. (canceled)

10. The curable mixture of claim 1, wherein the calcium silicate compound is calcium trisilicate.

11. The curable mixture of claim 10, wherein the curable mixture of ingredients comprises less than 20 wt % calcium silicate, based on the total weight of the curable mixture.

12. The curable mixture of claim 1, wherein the curable mixture of ingredients comprises between 0.1 wt. % to 30 wt. % filler, based on the total weight of the curable mixture of ingredients.

13. (canceled)

14. (canceled)

15. The curable mixture of claim 1, wherein the curable mixture of ingredients comprises between 40 wt % and 60 wt. % of a secondary carrier liquid.

16. The curable mixture of claim 1, wherein the secondary carrier liquid is water.

17. (canceled)

18. (canceled)

19. A kit, comprising:
a first container comprising a first part of a two-part curable composition, wherein the first part comprises:
(a) a calcium silicate compound,
(b) a filler material, and
(c) a first carrier liquid; and
a second container comprising a second part of the two-part curable composition, wherein the second part comprises a second carrier liquid.

20. (canceled)

21. (canceled)

22. The kit of claim 19, wherein the first carrier liquid is a non-aqueous liquid.

23. The kit of claim 19, wherein the second carrier liquid is an aqueous liquid.

24. (canceled)

25. The kit of claim 19, wherein the first part comprises a flow rate of about 50 g/min. to about 500 g/min. at 20 psi.

26. The kit of claim 19, wherein the second part comprises a viscosity between about 0.1 cps and 20 cps at 25° C.

27. A curable mixture of ingredients provided in two parts comprising
a first part comprising a flowable mixture comprising:
(a) a calcium silicate;
(b) a filler material; and
(c) a non-aqueous carrier liquid; and
a second part comprising an aqueous carrier liquid.

28. (canceled)

29. The curable mixture of claim 27, wherein a total weight percent of the non-aqueous carrier liquid and the aqueous carrier liquid is about 45 wt % to about 60 wt %.

30. The curable mixture of claim 27, wherein the non-aqueous carrier liquid comprises at least one of propylene glycol and poly(ethylene glycol).

31. (canceled)

32. The curable mixture of claim 27, comprising 1 wt % to 20 wt % tricalcium silicate.

33. (canceled)

34. (canceled)

35. (canceled)

36. The curable mixture of claim 27, comprising less than 40 wt. % filler.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. The curable mixture of claim 27, wherein the second aqueous carrier liquid has a viscosity of about 1 cps to about 30 cps at 25° C.

51. (canceled)

52. (canceled)

53. The curable mixture of claim 27, wherein the first part has a flow rate of about 50 g/min. to about 500 g/min. at 20 psi.

54. (canceled)

55. (canceled)

56. (canceled)

57. (canceled)

58. (canceled)

59. A method of filling a root canal or a tooth, comprising:
obtaining a first flowable part of a two-part curable composition that comprises:
(a) a calcium silicate compound,
(b) a filler material, and
(c) a first carrier liquid;
obtaining a second flowable part of the two-part curable composition, wherein the second part comprises a second carrier liquid;
combining the first and second flowable parts to form a final curable composition; and
positioning the final curable composition within the root canal or the tooth.

60. (canceled)

61. (canceled)

62. The method of claim 59, wherein the first carrier liquid is a non-aqueous liquid.

63. The method of claim 59, wherein the second carrier liquid is an aqueous liquid.
